SEQUENCE LISTING (10 sequences)

SEQ ID NO: 1 - DNA, 22 bp, ARTIFICIAL SEQUENCE
  SYNTHETIC OLIGONUCLEOTIDE
atttcaggtcccggatccggtg

SEQ ID NO: 2 - DNA, 6 bp, ARTIFICIAL SEQUENCE
  SYNTHETIC OLIGONUCLEOTIDE
aagctt

SEQ ID NO: 3 - DNA, 6 bp, ARTIFICIAL SEQUENCE
  SYNTHETIC OLIGONUCLEOTIDE
ggatcc

SEQ ID NO: 4 - DNA, 759 bp, ARTIFICIAL SEQUENCE
  SYNTHETIC OLIGONUCLEOTIDE
gnnccgcgtgtnnntcaagaacttgctcctcanntggatgttgcctttacttctaggcctgtacggaagtgttacttctgctctaaaagctgcggaattgtacccgcggccgggtgactgttaaaagtgcataccgccaaaagataaaatttgaaggccgcaattcccggggnatcgaaagagcctgctaaagcaaaaaagaagtcaccatgtcgtttactttgaccaacaagaacgtgattttcgttgccggtctgggaggcattggtctggacaccagcaaggagctgctcaagcgcgatcccgtcgttttacaacgtcgtgactgggnngccntnctgtgcncggntcttgntctnnnngncccncnngccccttncgtcngtnnccgcncagnnnngncgcgngtgtntcnttgcncttncngnnnntgcncnngctnnntnnggnnctgcngcngtnncctgntngngggtnncnctgtcnttgctnnnntncncnntngnttntnccgctcntnngctcgggtcgngtngntnnngcncntgnngggngtgncccnnnttgnnngtcngcctntnnggnnnctngtnnntcngnnntngggagtcgtttnntnntccctnggcnnccntcgcgncctcncggngcnggtnngctngcnnnnngtgnngngnnnntcgngtcgnctnttgtggtnnnnngcnnggnnnggtcncncnncgcccctnnccctngcncggctngcntcntcngncn

SEQ ID NO: 5 - DNA, 744 bp, ARTIFICIAL SEQUENCE
  SYNTHETIC OLIGONUCLEOTIDE
acatgaacttgctcctcactggatgtttgcctttacttctaggcctgtacggaagtgttacttctgctctaaaagctgcggaattgtacccgcggccgggtgactgttaaaagtgcataccgccaaaagataaaatttgaaggccgcaattcccggggatcgaaagagcctgctaaagcaaaaaagaagtcaccatgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcnttgcctggtttccggcaccanaancgntgccgnnnannnnncnnnnctccnntnnnnnnnnnncnnnncnnncnnnnnccnnnnnnnnnncnnnnccnnnnnnnnnnnccnnnnnnnnnnncnnnnncnnnnnnnnnnnnnccnnnncnnnnccnnnnnnnnnnnnnnnnnnnnnccnnncncnnnnnnnnnnnnncnnnnnnnncnnnnnnncnnnnnnncnnnnnnncnnnnnncnnnnnnnnnnnnnnnnnnnnnnncnnnnncnnnnnnnnnnnnnnnnnnnnnnccccnnnnnnn

SEQ ID NO: 6 - DNA, 715 bp, ARTIFICIAL SEQUENCE
  SYNTHETIC OLIGONUCLEOTIDE
taaagaactgctcctcagntggatgttgcctttacttctaggcctgtacggaagtgttacttctgctctaaaagctgcggaattgtacccgcggccgggtgactgttaaaagtgcataccgccaaaagataaaatttgaaggccgcaattcccggggatcgaaagagcctgctaaagcaaaaaagaagtcaccatgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgncagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgangccgatactgtcgtcgtcccctcanactggcagatgcacggttacgatgcgcccatctacaccaacgtganctatcccattacggtcatccgccgtttgtnccacgnanaatccgacggnttgtactcgctcacatnnatgttnatnaancngncnannnangncanancnantatnnnnntgncntnanncgnngttcatcngtgnnn

SEQ ID NO: 7 - DNA, 710 bp, ARTIFICIAL SEQUENCE
  SYNTHETIC OLIGONUCLEOTIDE
acgggcagtgatgatcgactcgactntaagtagttcatcaagcctgcgcctctctttaattgaaacagctgctgcccgagtaataagagtatccgatattcctacagcaattttagataaagttgagatcgaacatggcacaataaccatagcatcaaaaagaaaacttcctgaactaacaggtgcagtaaaatcatcatcagaatatgtatagcttgccagggaacttaagtatgatggatcttgattcatctcatacttcattacctttttagcattctctgaaattattagatgtacctcattttgtcctaggttttcaagtagcttaactgcatatggtataccactggcccctgatactcctacaactattctcatccaaaatcatatcacttcttcctattccaacatacaggctcttgatgcaggagctattataacgccattttccgatagagagagcatgtttttaagatcaatggaacttaaaggcatttcccttggtacgatgatgagcctgcgcctctctttaattgaaacagctgctgcccgagtaataagagtatccgatattcctacagcaatttgagataaagttgagatcgaacatggcacaataaccatagcatcaaaaagaaaacttcctgaactaacaggtgcagtaaaaatcatccatcagaatatgtttagcttgccagggaacttag

SEQ ID NO: 8 - DNA, 53 bp, ARTIFICIAL SEQUENCE
  SYNTHETIC OLIGONUCLEOTIDE
cnctcagatcctctgccacaagtttggacaaaatgaacancactgtaataagt

SEQ ID NO: 9 - DNA, 716 bp, ARTIFICIAL SEQUENCE
  SYNTHETIC OLIGONUCLEOTIDE
agacggcagtgatgatcgactcgactncaagtagttcatcaagcctgcgcctctctttaattgaaacagctgctgcccgagtaataagagtatccgatattcctacagcaattttagataaagttgagatcgaacatggcacaataaccatagcatcaaaaagaaaacttcctgaactaacaggtgcagtaaaatcatcatcagaatatgtatagcttgccagggaacttaagtatgatggatcttgattcatctcatacttcattacctttttagcattctctgaaattattagatgtacctcattttgtcctaggttttcaagtagcttaactgcatatggtataccactggcccctgatactcctacaactattctcatccaaaatcatatcacttcttcctattccaacatacaggctcttgatgcaggagctattataacgccattttccgatagagagagcatgtttttaagatcaatggaacttaaaggcatttcccttggtacgatgatgagcctgcgcctctctttaattgaaacagctgctgcccgagtaataagagtatccgatattcctacagcaattttagataaaggttgagatcgaacatgggacaattaaccataggntcaaaaaggaaaacttctggaactaaacaggtgcagtaaaatcatccatcagaatatgtttagctggccagggaaccaaaat

SEQ ID NO: 10 - DNA, 726 bp, ARTIFICIAL SEQUENCE
  SYNTHETIC OLIGONUCLEOTIDE
gcaacggcagtgatgattcgagctcgacggtttatagtcatcaaagcctgcgcctctctttaattgaaacagctgctgcccgagtaataagagtatccgatattcctacagcaattttagataaagttgagatcgaacatggcacaataaccatagcatcaaaaagaaaacttcctgaactaacaggtgcagtaaaatcatcatcagaatatgtatagcttgccagggaacttaagtatgatggatcttgattcatctcatacttcattacctttttagcattctctgaaattattagatgtacctcattcttgtcctaggttttcaagtagcttaactgcatatggtataccactggcccctgatactcctacaactattctcatccaaaatcatatcacttcttcctattccaacatacaggctcttgatgcaggagctattataacgccattttccgatagagagagcatgtttttaagatcaatggaacttaaaggcatttcccttggtacgatgatgagcctggcgccctctctttaattgaaacagctgctgcccgagtaataagagtatcccgatattcctacagcaattttagataaaagttgagattcgaaccatggggacaataacattaggctccaaaaacgaaaaacttgctgaactaacaggtgcagtaaaatcatcatcaggatatgtatagcttgccagggaactaagtat

(19) United States
(12) Patent Application Publication
(10) Pub. No.: US 2005/0239087 A1
(43) Pub. Date: Oct. 27, 2005

(54) Multiply-primed amplification of nucleic acid sequences

(76) Inventors: Haiguang Xiao, Princeton, NJ (US); Scott Hamilton, Phillipsburg, NJ (US)

Correspondence Address: Amersham Biosciences Corp, 800 Centennial Avenue, Piscataway, NJ, 08855, US

(21) Appl. No.: 10/835,140

(22) Filed: Apr. 29, 2004

Related U.S. Application Data
(60) Provisional application No. 60/466,513, filed on Apr. 29, 2003.

(51) Int. Cl. C12Q 1/68; C12P 19/34

(52) U.S. Cl. 435/6; 435/91.2

(57) ABSTRACT

Improved processes for the amplification of target DNA sequences in the form of single or double stranded DNA molecules, especially those present in colony and plaque extracts, using multiple specific and/or random sequence oligonucleotide primers are disclosed along with methods for detecting such amplified target sequences wherein some or all of the deoxyribonucleotides are replaced by deoxyribonucleotide analogues that reduce the Tm of the amplified product. The product of this amplification is used for DNA sequencing and other analyses that involve hybridization. Kits containing components for use in the invention is also described. Also described are further uses of this amplified DNA in sequencing, single base substitution detection, modifying the restriction enzyme fragmentation patterns and other molecular biology applications.

### CROSS-REFERENCE TO RELATED APPLICATIONS

This application claims priority to U.S. provisional patent application No. 60/466,513 filed on Apr. 29, 2003, the entire disclosure of which is incorporated herein by reference in its entirety.

### BACKGROUND OF THE INVENTION

1. Field of the Invention

The present invention relates to improved processes for DNA amplification by multiply primed rolling circle and multiple displacement amplification so as to provide modified products. The amplification process is carried out using various nucleotide analogs giving the product improved properties, particularly for further analysis by sequencing or other methods.

2. Description of Related Art

Several useful methods have been developed that permit amplification of nucleic acids. Most were designed around the amplification of selected DNA targets and/or probes, including the polymerase chain reaction (PCR), ligase chain reaction (LCR), self-sustained sequence replication (3SR), nucleic acid sequence based amplification (NASBA), strand displacement amplification (SDA), and amplification with Q.β. replicase (Birkenmeyer and Mushahwar, J. Virological Methods, 35:117-126 (1991); Landegren, Trends Genetics, 9:199-202 (1993)).

In addition, several methods have been employed to amplify circular DNA molecules such as plasmids or DNA from bacteriophage such as M13. One has been propagation of these molecules in suitable host strains of E. coli, followed by isolation of the DNA by well-established protocols (Sambrook, J., Fritsch, E. F., and Maniatis, T. Molecular Cloning, A Laboratory Manual, 1989, Cold Spring Harbor Laboratory Press, Cold Spring Harbor, N.Y.). PCR has also been a frequently used method to amplify defined sequences in DNA targets such as plasmids and DNA from bacteriophage such as M13 (PCR Protocols, 1990, Ed. M. A. Innis, D. H. Gelfand, J. J. Sninsky, Academic Press, San Diego.) Some of these methods suffer from being laborious, expensive, time-consuming, inefficient, and lacking in sensitivity.

As an improvement on these methods, linear rolling circle amplification (LRCA) uses a primer annealed to a circular target DNA molecule and DNA polymerase is added. The amplification target circle (ATC) forms a template on which new DNA is made, thereby extending the primer sequence as a continuous sequence of repeated sequences complementary to the circle but generating only about several thousand copies per hour. An improvement on LRCA is the use of exponential RCA (ERCA), with additional primers that anneal to the replicated complementary sequences to provide new centers of amplification, thereby providing exponential kinetics and increased amplification. Exponential rolling circle amplification (ERCA) employs a cascade of strand displacement reactions, also referred to as HRCA (Lizardi, P. M. et al. Nature Genetics, 19, 225-231 (1998)). However, ERCA is limited to the use of just a single primer P1 annealed to the circular DNA target molecule, to the need to know the specific DNA sequence for the primer P1, and for the need of the circular DNA target molecule to be a single-stranded DNA circle.

In U.S. Pat. No. 6,323,009 (see also U.S. patent application Ser. No. 09/920,571), a means of amplifying target DNA molecules is introduced. This method is of value because such amplified DNA is frequently used in subsequent methods including DNA sequencing, cloning, mapping, genotyping, generation of probes for hybridization experiments, and diagnostic identification.

The methods of the U.S. Pat. No. 6,323,009 patent (referred to herein as Multiply Primed Amplification—MPA) avoid such disadvantages by employing procedures that improve on the sensitivity of linear rolling circle amplification by using multiple primers for the amplification of individual target circles. The MPA method has the advantage of generating multiple tandem-sequence DNA (TS-DNA) copies from each circular target DNA molecule. In addition, MPA has the advantages that in some cases the sequence of the circular target DNA molecule may be unknown while the circular target DNA molecule may be single-stranded (ssDNA) or double-stranded (dsDNA or duplex DNA). Another advantage of the MPA method is that the amplification of single-stranded or double-stranded circular target DNA molecules may be carried out isothermally and/or at ambient temperatures. Other advantages include being highly useful in new applications of rolling circle amplification, low cost, sensitivity to low concentration of target circle, flexibility, especially in the use of detection reagents, and low risk of contamination.

The MPA method can improve on the yield of amplified product DNA by using multiple primers that are resistant to degradation by exonuclease activity that may be present in the reaction. This has the advantage of permitting the primers to persist in reactions that contain an exonuclease activity and that may be carried out for long incubation periods. The persistence of primers allows new priming events to occur for the entire incubation time of the reaction, which is one of the hallmarks of ERCA and has the advantage of increasing the yield of amplified DNA.

The MPA method allows for the first time “in vitro cloning”, i.e. without the need for cloning into an organism, of known or unknown target DNAs enclosed in circles. A padlock probe may be used to copy the target sequence into a circle by the gap fill-in method (Lizardi, P. M. et al. Nature Genetics, 19,225-231 (1998)). Alternatively, target sequences can be copied or inserted into circular ssDNA or dsDNA by many other commonly used methods. The MPA amplification overcomes the need to generate amplified yields of the DNA by cloning in organisms.

The MPA method is an improvement over LRCA in allowing increased rate of synthesis and yield. This results from the multiple primer sites for DNA polymerase extension. Random primer MPA also has the benefit of generating double stranded products. This is because the linear ssDNA products generated by copying of the circular template will themselves be converted to duplex form by random priming of DNA synthesis. Double stranded DNA product is advantageous in allowing for DNA sequencing of either strand and for restriction endonuclease digestion and other methods used in cloning, labeling, and detection.

It is also expected that strand-displacement DNA synthesis may occur during the MPA method resulting in an exponential amplification. This is an improvement over conventional ERCA, also termed HRCA (Lizardi et al. (1998)) in allowing for the ability to exponentially amplify very large linear or circular DNA targets. The amplification of large circular DNA, including bacterial artificial chromosomes (BACs), has been reduced to practice using the MPA method.

Methods have published for whole genome amplification using degenerate primers (Cheung, V. G. and Nelson, S. F. Proc. Natl. Acad. Sci. USA, 93, 14676-14679 (1996) and random primers (Zhang, L. et al., Proc. Natl. Acad. Sci. USA, 89, 5847-5851 (1992) where a subset of a complex mixture of targets such as genomic DNA is amplified. Reduction of complexity is an objective of these methods. A further advantage of the MPA method is that it amplifies DNA target molecules without the need for “subsetting”, or reducing the complexity of the DNA target.

The MPA method rapidly amplifies every sample of DNA used with it, the double-stranded product has all the same sequences as the original sample. Except for the fact that it contains tandemly-repeated copies of the DNA with numerous initiation (priming) sites, the physical properties of the product DNA are much like those of the starting template.

Dierick, H. et al., Nucleic Acids Resh 21, 4427-8 (1993) describe PCR amplification of a 560 bp sequence using dGTP analogs dITP or 7-deaza-dGTP. They report that if they subsequently separate the PCR product strands using magnetic beads and sequence them, improved sequences are obtained when PCR is performed using the dGTP analogs, particularly dITP. Presumably, this is the result of altered physical properties of the product DNA strands although the length of the strands was confirmed. This method, however, will only work for situations in which two PCR primer sequences can be specified for the region to be sequenced, is limited to sequences of at most about 1000 nucleotides that can readily be amplified by PCR, and requires thermal cycling for amplification.

Accordingly, there is a need for amplification methods that lack the limitations of PCR. For example, in PCR, the fraction of substitution of one nucleotide for another may be limited, particularly for substituting dITP for dGTP. In addition, the fidelity of PCR when using dITP is known to be compromised. These concerns are addressed in greater detail below.

### SUMMARY OF THE INVENTION

Accordingly, it is the object of the invention to provide a new method of amplification that can be used for any DNA even if the sequence is not known, can provide for complete or near-complete substitution of nucleotide analogs for the usual nucleotides, and which can be carried out isothermally at temperatures down to 0° C. This and other objectives were met by the present invention, which employs modified MPA (mMPA) using non-natural nucleotides to prepare DNA that may be used for sequenceing or other downstream analysis purposes.

The present invention relates to a process for the enhanced amplification of DNA targets using either specific or random primers. In a specific embodiment, this aspect of the invention employs multiple primers (specific or random, exonuclease-sensitive or exonuclease-resistant) annealed to the target DNA molecules to increase the yield of amplified product from RCA. Multiple primers anneal to multiple locations on the target DNA and extension by polymerase is initiated from each location. In this way multiple extensions are achieved simultaneously from the target DNA. The extension process is carried out in the presence of one or more nucleotide analogs, optionally in the presence of all four normal nucleotides. The nucleotide analogs confer unusual properties to the product DNA without changing its sequence content.

The use of multiple primers is achieved in several different ways. It is achieved by using two or more specific primers that anneal to different sequences on the target DNA, or by having one given primer anneal to a sequence repeated at two or more separate locations on the target DNA, or by using random or degenerate primers, which can anneal to many locations on the target DNA.

In a particularly advantageous embodiment, dITP is substituted for some or all of the dGTP in the amplification reaction mixture. The addition of the dITP, it has been found, does not deleteriously affect the MPA reaction, producing significant quantities of amplified nucleic acid.

There is, however, a class of DNA sequences which are characteristically difficult to sequence using current dye-terminator cycle-sequencing methods which also make use of dITP to prevent certain electrophoresis artifacts. The members of this class of sequences all have low-complexity, highly G and C rich repeat sequences which have symmetry that suggests the sequences are self-complimentary, capable of forming hairpin-style secondary structures. It is likely that during the DNA synthesis required for DNA sequencing, the newly-synthesized DNA strand (containing dI) can be displaced at these repeat sequences by the template DNA strand containing dG which forms stronger base-pairs particularly during cycle sequencing at relatively high temperatures. We have found that substituting dI for dG in the template strand eliminates this particular class of extremely difficult-to-sequence DNAs and that this substitution is quite facile using mMPA to prepare the template DNA for sequence analysis.

In another embodiment, the deoxyribonucleoside-5′-triphosphates (dNTPs) used in the MPA reaction may be substituted by their analogs that upon incorporation reduce the Tm of the amplified product. For example, dGTP may be substituted by 7-deaza-dGTP (Seela, U.S. Pat. No. 4,804,748 and U.S. Pat. No. 5,480,980), 7-deaza-dITP, 7-substituted-7-deaza-dITP or dGTP (Fuller, McDougall & Kumar, GB 2323357A). Similarly, dATP may be substituted by 7-deaza-dATP or related analogs, dCTP may be substituted by N4-alkyl-dCTP (Nucleic Acids Res. 1993, 21, 2709-14), 5-alkyl-dCTP or related analogs, and dTTP may be substituted by 5-substituted-DTTP.

In some embodiments, the primers for MPA contain nucleotides, including all types of modified nucleotides, which may serve to make the primers resistant to enzyme degradation. Enzyme degradation may be caused by a specific exonuclease such as the 3′-5′ exonuclease activity associated with DNA polymerase or by a non-specific, contaminating exonuclease.

The objects and features of the invention are more fully apparent following review of the detailed description of the invention in conjunction with the accompanying drawings.

### BRIEF DESCRIPTION OF THE DRAWINGS

FIG. 1 shows an electropherogram of a DNA sequencing reaction using DYEnamic ET-terminator kit (Amersham Biosciences Inc.) with DNA amplified by standard MPA from 2 ng of pNASSβ DNA as the template and 5 pmol of MHXP primer.

FIG. 2 is an electropherogram of a DNA sequencing reaction using DYEnamic ET-terminator kit (Amersham Biosciences Inc.) with DNA amplified by modified MPA (with 0.4 mM dITP alone) from 2 ng of pNASSβ DNA as the template and 5 pmol of MHXP primer.

FIG. 3 is an electropherogram of a DNA sequencing reaction using DYEnamic ET-terminator kit (Amersham Biosciences Inc.) with DNA amplified by modified MPA (with 0.8 mM dITP and 0.05 mM dGTP) from 2 ng of pNASSβ DNA as the template and 5 pmol of MHXP primer.

FIG. 4 is an electropherogram of a DNA sequencing reaction using DYEnamic ET-terminator kit (Amersham Biosciences Inc.) with DNA amplified by standard MPA from 1 μl glycerol stock of a random library of T. Volcanium DNA as the template. Reactions were cycled at normal temperature (30 times at 95° C., 20 seconds, 50° C., 30 seconds and 60° C., 60 seconds).

FIG. 5 is an electropherogram of a DNA sequencing reaction using DYEnamic ET-terminator kit (Amersham Biosciences Inc.) with DNA amplified by standard MPA from 1 μl glycerol stock of a random library of T. Volcanism DNA as the template. Reactions were cycled at low temperature (30 times at 82° C., 20 seconds, 40° C., 30 seconds and 50° C., 60 seconds)

FIG. 6 is an electropherogram of a DNA sequencing reaction using DYEnamic ET-terminator kit (Amersham Biosciences Inc.) with DNA amplified by modified MPA (with 0.8 mM dITP and 0.05 mM dGTP from 1 μl glycerol stock of a random library of T. Volcanium DNA as the template. Reactions were cycled at normal temperature (30 times at 95° C., 20 seconds, 50° C., 30 seconds and 60° C., 60 seconds)

FIG. 7 is an electropherogram of a DNA sequencing reaction using DYEnamic ET-terminator kit (Amersham Biosciences Inc.) with DNA amplified by modified MPA (with 0.8 mM dITP and 0.05 mM dGTP from 1 μl glycerol stock of a random library of T. Volcanium DNA as the template. Reactions were cycled at low temperature (30 times at 82° C., 20 seconds, 40° C., 30 seconds and 50° C., 60 seconds)

### DETAILED DESCRIPTION OF THE INVENTION

The present invention pertains to analysis of DNA and in particular to analyses that depend on the sequence of DNA, often used for determining genotype as well as original sequence information. It also pertains to amplification of DNA sequences. Amplification means synthesis of new strands of DNA which have complimentary sequence to the original, preserving the original sequence information. While some amplification methods such as polymerase chain reaction (PCR) are highly specific and yield amplified products of defined length, others are general, amplifying all the DNA sequences present in a sample yielding products that vary in length yet still contain the original sequence information. An example of this latter kind of amplification is MPA as described in U.S. Pat. No. 6,323,009.

This invention also pertains to DNA sequencing, which is defined as a method for determining the nucleotide base sequence of a DNA molecule comprising the steps of incubating the nucleic acid molecule with an oligonucleotide primer, a plurality of deoxynucleoside triphosphates, at least one chain terminating agent, and a DNA polymerase under conditions in which the primer is extended until the chain terminating agent is incorporated. The products are separated according to size, detected and whereby at least a part of the nucleotide base sequence of the original DNA molecule can be determined (see, for example U.S. Pat. No. 5,639,608).

A more advantageous sequencing method is cycle sequencing with dideoxynucleotide terminators. Cycle sequencing involves multiple rounds of DNA synthesis carried out from the same template using an oligonucleotides primer. The newly synthesized strand is removed from the template strand after each synthesis cycle by heat denaturation; this amplifies the number of strands produced in the sequencing process and allows much smaller amounts of DNA template to be sequenced (U.S. Pat. No. 5,614,365). A particularly useful way of performing cycle sequencing is with thermally stable DNA polymerase and fluorescent-labeled dideoxynucleotide terminators (for example U.S. Pat. No. 5,366,860). This, most popular method of sequencing typically makes use of dITP to eliminate electrophoresis artifacts, and four distinct fluorescent labels for the four nucleotide bases.

The polymerase chain reaction (PCR) is defined as a process for amplifying at least one specific nucleic acid sequence contained in a nucleic acid or a mixture of nucleic acids wherein each nucleic acid consists of two separate complementary strands. First, the strands are combined with two oligonucleotide primers, for the specific sequence being amplified, under conditions such that the extension product synthesized from one primer, when it is separated from its complement, can serve as a template for synthesis of the extension product of the other primer. The primers are extended using DNA polymerase then the extension products denatured by heating from the templates on which they were synthesized to produce single-stranded molecules. Upon cooling to an annealing temperature, the single-stranded molecules generated anneal with the primers and are again extended by DNA polymerase. The process is repeated one or more times resulting in exponential amplification of the sequences “between” the priming sites U.S. Pat. No. 4,683,202.

Single Strand Confirmation Polymorphism (SSCP) is a process that can be used for the detection of polymorphisms (Orita et al, PNAS 86(8) April 1989 2766-70; Lessa-et al. Mol Ecol 2(2) p. 119-29 April 1993). Essentially, labeled, denatured fragments of DNA are applied to a non-denaturing electrophoresis gel. If polymorphisms (sequence variants) exist in the fragment, more than one band may be observed on the gel because the conformation of the single-stranded fragments differ with different sequences.

Hybridization is a technique of using the natural tendency for nucleic acids to bind specifically to other nucleic acid strands with complimentary sequence. Virtually all molecular biology experiments feature hybridization, for example the sequencing primer hybridizes with the sequencing template. Similarly the PCR primers hybridize with the desired template strands. More general hybridization experiments may involve hybridization of an immobilized nucleic acid with a soluble, labeled or tagged nucleic acid in techniques variously called “Southern” hybridizations (Southern, E., J Mol. Biol. 1975 98(3):503-17), “Northern” hybridizations (Alwine et. Al, Proc Natl Acad Sci USA. 1977; 74(12):5350-4) and more recent microarray hybridizations (see for example WO9210588). The invention relates to the use of multiple primers in nucleic acid sequence amplification as a means of greatly amplifying DNA synthesis and providing greatly increased amounts of DNA for detection of specific nucleic acid sequences contained in, for example, a target DNA. While previous methods have often employed targets of substantial complexity, the present invention utilizes relatively simple targets, such as simple plasmid, cosmid and bacterial artificial chromosome (BAC) targets. The target DNA useful in the present invention also includes linear DNA, even high molecular weight linear DNA.

The present invention further relates to the discovery that the replacement of some or all of the normal nucleotides (e.g. dGTP) within the amplification reaction mix by modified nucleotide analogs (e.g. dITP, 2′-deoxy Inosine triphosphate) produces an amplification product with significantly enhanced properties, including ability to be sequenced and ability to hybridize at altered temperature.

In addition, while other methodologies have attempted to amplify random subsets of substantially complex target DNA molecules (for example, a nucleic acid, including either DNA or RNA, whose presence in a sample is to be detected or whose sequence is to be amplified, such as for use in subsequent methods or procedures, or whose presence in said sample determines the identity of one or more other nucleic acids whose sequence(s) is/are to be amplified) to generate a less complex set of amplified materials, the present invention relates to the amplification of all the sequences present in the target, with no attempts at any reduction in sequence complexity.

In one embodiment one can provide a premix, such as in the form of a kit, comprising a polymerase, even including more than one polymerase, nuclease-protected oligonucleotide primers, such as random-sequence hexamers, the required nucleoside triphosphates, an appropriate buffer, optionally a pyrophosphatase, and other potentially desirable components, either with each such component in a separate vial or mixed together in different combinations so as to form a total of one, two, three, or more separate vials and, for example, a blank or buffer vial for suspending an intended target nucleic acid for use in the amplification process. One embodiment of the present invention comprises a kit for amplifying DNA sequences comprising nuclease-resistant random primers, a DNA polymerase and the four deoxyribonucleoside triphosphates (dNTPs),. In a separate embodiment, said DNA polymerase has 3′-5′ exonuclease activity. In a preferred embodiment, said DNA polymerase is φ29 DNA polymerase. In a most preferred embodiment, at least one of the normal dNTPs is replaced, in whole or in part, by an analog whose presence in the product DNA confers some advantageous property to said product DNA or to subsequent processes such as sequence-dependent analyses.

In a specific application of such an embodiment, there is provided a process whereby a sample of nucleic acid, such as a DNA, is suspended in a buffer, such as TE buffer, and then heated, cooled, and then contacted with the components recited above, either sequentially or by adding such components as the aforementioned premix with the conditions of temperature, pH and the like subsequently adjusted, for example by maintaining such combination at 10° C.

In addition, the conditions used in carrying out the processes disclosed according to the present invention may vary during any given application. Thus, by way of non-limiting example, the primers and target DNA may be added under conditions that promote hybridization and the DNA polymerase and nucleoside triphosphates added under different conditions that promote amplification without causing denaturation of the primer-target complexes that act as substrates for the polymerase or polymerases.

In one embodiment, the present invention relates to a process as described herein wherein the target DNA binds to, or hybridizes to, at least 3, 4, 5, even 10, or more primer oligonucleotides, each said primer producing, under appropriate conditions, a separate tandem sequence DNA molecule. Of course, because the sequences of the tandem sequence DNAs (TS-DNAs) are complementary to the sequences of the target DNA, which act as template, the TS-DNA products will all have the same sequence as the target DNA, regardless of the sequence of the primers and the nucleotide content of the TS-DNA product will be determined by the mixture of nucleotides or nucleotide analogs used for the amplification subject to the selective power of the DNA polymerase or polymerases used in the amplification process.

The oligonucleotide primers useful in the processes of amplification can be of any desired length. For example, such primers may be of a length of from at least 2 to about 30 to 50 nucleotides long, preferably about 2 to about 35 nucleotides in length, most preferably about 5 to about 10 nucleotides in length, with hexamers and octamers being specifically preferred embodiments. Such multiple primers as are used herein may equally be specific only, or random-sequence only, or a mixture of both, with random primers being especially useful and convenient to form and use.

Amplification target DNA useful in the processes of the present invention are DNA or RNA molecules, either single or double stranded, including DNA-RNA hybrid molecules generally containing between 40 to 10,000 nucleotides. However, it is expected that there will be no upper limit to the size of the target, particularly when using short, random-sequence primers. Where the target is a duplex, such numbers are intended to refer to base pairs rather than individual nucleotide residues. The target templates useful in the processes disclosed herein may have functionally different portions, or segments, making them particularly useful for different purposes. At least two such portions will be complementary to one or more oligonucleotide primers and, when present, are referred to as a primer complementary portions or sites. Amplification targets useful in the present invention include, for example, those derived directly from such sources as a bacterial colony, a bacteriophage, a virus plaque, a yeast colony, a baculovirus plaque, as well as transiently transfected eukaryotic cells. Such sources may or may not be lysed prior to obtaining the targets. Where such sources have been lysed, such lysis is commonly achieved by a number of means, including where the lysing agent is heat, an enzyme, the latter including, but not limited to, enzymes such as lysozyme, helicase, glucylase, and zymolyase, or such lysing agent may be an organic solvent.

In MPA, amplification occurs with each primer, thereby forming a concatemer of tandem repeats (i.e., a TS-DNA) of segments complementary to the primary ATC (or ATC) being replicated by each primer. Thus, where random primers are used, many such TS-DNAs are formed, one from each primer, to provide greatly increased amplification of the corresponding sequence since the nucleotide sequence, or structure, of the product depends only on the sequence of the template and not on the sequences of the oligonucleotide primers, whether the latter are random or specific or a mixture of both.

The amplification method used in the present invention is distinct from published modified PCR methods (see for example Cheung, V. G. and Nelson, S. F. Proc. Natl. Acad. Sci. USA, 93, 14676-14679 (1996); and Zhang, L. et al., Proc. Natl. Acad. Sci. USA, 89, 5847-5851 (1992)) by facilitating use of random or multiple primers in an amplification of linear DNA target with a DNA polymerase, such as φ29 DNA polymerase as a preferred enzyme for this reaction, along with exonuclease-resistant primers (as described below). Therefore, the present invention includes a method for the amplification of linear DNA targets, including high molecular weight DNAs, as well as genomic and cDNAs, that takes advantage of the characteristics of φ29 DNA polymerase and the exonuclease-resistant primers that are compatible with the 3′-5′ exonuclease activity associated with φ29 DNA polymerase and wherein said linear DNA target may be used instead of or in addition to circular DNA.

Where duplex circles are employed, amplification will commonly occur from both strands as templates. Simultaneous amplification of both circles may or may not be desirable. In cases where the duplex circles are to be further employed in reactions designed to sequence the DNA of said circles, amplification of both strands is a desirable feature and so the duplex circles can be directly employed without further processing (except for formation of a nick if needed). However, for other uses, where co-temporal amplification of both strands is not a desired feature, it is well within the skill of those in the art to denature and separate the strands prior to amplification by the processes of the present invention or, alternatively, to employ multiple specific primers that contain sequences complementary to only one of the two strands of the duplex circular template. No doubt other useful strategies will immediately occur to those of skill in the art and need not be further described herein.

In some circumstances it may be desirable to quantitatively determine the extent of amplification occurring. In such instances, the amplification step of the present invention works well with any number of standard detection schemes, such as where special deoxynucleoside triphosphates (dNTPs) are utilized that make it easier to do quantitative measurements. The most common example is where such nucleotide substrates are radiolabeled or have attached thereto some other type of label, such as a fluorescent label or the like. These are typically used in trace amounts so as to minimally disturb the composition of the product DNA. Again, the methods that can be employed in such circumstances are many and the techniques involved are standard and well known to those skilled in the art. Thus, such detection labels include any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. General examples include radioactive isotopes, fluorescent molecules, phosphorescent molecules, enzymes, antibodies, and ligands. The use of such trace amounts of labeled or tagged nucleotides is considered distinct from the use of sufficient quantities of nucleotide analogs to significantly alter the physical properties of the product DNA sush as changing the melting temperature of the product DNA by as much as 1° C. to as much as 20° C. or more.

Examples of suitable fluorescent labels include Cy Dyes such as Cy2, Cy3, Cy3.5, Cy5, And Cy5.5, available from Amersham Pharmacia Biotech (U.S. Pat. No. 5,268,486). Further examples of suitable fluorescent labels include fluorescein, 5,6-carboxymethyl fluorescein, Texas red, nitrobenz-2-oxa-1,3-diazol-4-yl (NBD), coumarin, dansyl chloride, and rhodamine. Preferred fluorescent labels are fluorescein (5-carboxyfluorescein-N-hydroxysuccinimide ester) and rhodamine (5,6-tetramethyl rhodamine). These can be obtained from a variety of commercial sources, including Molecular Probes, Eugene, Oreg. and Research Organics, Cleveland, Ohio.

Labeled nucleotides are a preferred form of detection label since they can be directly incorporated into the products of amplification during synthesis. Examples of detection labels that can be incorporated into amplified DNA include nucleotide analogs such as BrdUrd (Hoy and Schimke, Mutation Research, 290:217-230 (1993)), BrUTP (Wansick et al., J. Cell Biology, 122:283-293 (1993)) and nucleotides modified with biotin (Langer et al., Proc. Natl. Acad. Sci. USA, 78:6633 (1981)) or with suitable haptens such as digoxygenin (Kerkhof, Anal. Biochem., 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label is Biotin-16-uridine-5′-triphosphate (Biotin-16-dUTP, Boehringher Mannheim). Radiolabels are especially useful for the amplification methods disclosed herein. Thus, such dNTPs may incorporate a readily detectable moiety, such as a fluorescent label as described herein.

The methods of the present invention provide high amplification rates due to multiple priming events being induced on molecules that are targets for amplification. Thus, the rate and extent of amplification is not limited to that accomplished by a single DNA polymerase copying the DNA circle. Instead, multiple DNA polymerases are induced to copy each template circle simultaneously, each one initiating from one of the primers. It is this feature that provides a unique advantage of the present method and compensates for decreased synthesis rate caused by the use of nucleotide analogs such as dITP in place of dGTP.

Exonuclease-resistant primers useful in the methods disclosed herein may include modified nucleotides to make them resistant to exonuclease digestion. For example, a primer may possess one, two, three or four phosphorothioate linkages between nucleotides at the 3′ end of the primer.

Thus, in some embodiments, the amplification step relates to processes wherein the primers contain at least one nucleotide that makes the primer resistant to degradation, commonly by an enzyme, especially by an exonuclease and most especially by 3′-5′-exonuclease activity. In such an embodiment, at least one nucleotide may be a phosphorothioate nucleotide or some modified nucleotide. Such nucleotide is commonly a 3′-terminal nucleotide but the processes of the present invention also relate to embodiments wherein such a nucleotide is located at other than the 3′-terminal position and wherein the 3′-terminal nucleotide of said primer can be removed by 3′-5′-exonuclease activity.

Attachment of target templates or oligonucleotide primers to solid supports may be advantageous and can be achieved through means of some molecular species, such as some type of polymer, biological or otherwise, that serves to attach said primer or target template to a solid support. Such solid-state substrates useful in the methods of the invention can include any solid material to which oligonucleotides can be coupled. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, fibers, woven fibers, shaped polymers, particles and microparticles. A preferred form for a solid-state substrate is a glass slide or a microtiter dish (for example, the standard 96-well dish). Preferred embodiments utilize glass or plastic as the support. For additional arrangements, see those described in U.S. Pat. No. 5,854,033.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994).

Oligonucleotide primers useful in the present invention can be synthesized using established oligonucleotide synthesis methods. Methods of synthesizing oligonucleotides are well known in the art. Such methods can range from standard enzymatic digestion followed by nucleotide fragment isolation (see for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods in Enzymology, 65:610-620 (1980), (phosphotriester method). Protein nucleic acid molecules can be made using known methods such as those described by Nielsen et al., Bioconjugate. Chem. 5:3-7 (1994).

Methods for the synthesis of primers containing exonuclease-resistant phosphorothioate diesters by chemical sulfurization are well-established. The solid phase synthesis of random primers employs one or several specifically placed internucleotide phosphorothioate diesters at the 3′-end. Phosphorothioate triesters can be introduced by oxidizing the intermediate phosphite triester obtained during phosphoramidite chemistry with 3H-1,2-benzodithiol-3-one 1,1 dioxide.sup.1,2 or Beaucage reagent to generate pentavalent phosphorous in which the phosphorothioate triester exists as a thione. The thione formed in this manner is stable to the subsequent oxidation steps necessary to generate internucleotidic phosphodiesters. (Iyer, R. P., Egan, W., Regan, J. B., and Beaucage, S. L. J. Am. Chem. Soc., 112: 1253 (1990), and Iyer, R. P., Philips, L. R., Egan, W., Regan, J. B., and Beaucage, S. L. J. Org. Chem., 55: 4693 (1990))

Many of the oligonucleotides described herein are designed to be complementary to certain portions of other oligonucleotides or nucleic acids such that hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

DNA polymerases useful in the isothermal amplification step are referred to herein as amplification DNA polymerases. For amplification, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the target template. A 5′ to 3′ exonuclease activity, if present, might result in the destruction of the synthesized strand. It is also preferred that DNA polymerases for use in the disclosed method are highly processive. The suitability of a DNA polymerase for use in the disclosed method can be readily determined by assessing its ability to carry out amplification. Preferred amplification DNA polymerases, all of which have 3′,5′-exonuclease activity, are bacteriophage .φ0.29 DNA polymerase (U.S. Pat. Nos. 5,198,543 and 5,001,050 to Blanco et al.), phage M2 DNA polymerase (Matsumoto et al., Gene 84:247 (1989)), phage PRD1 DNA polymerase (Jung et al., Proc. Natl. Aced. Sci. USA 84:8287 (1987), and Zhu and Ito, Biochim. Biophys. Acta. 1219:267-276 (1994)), VENT.TM. DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), and T4 DNA polymerase holoenzyme (Kaboord and Benkovic, Curr. Biol. 5:149-157 (1995)). .φ0.29 DNA polymerase is most preferred. Equally preferred polymerases include native T7 DNA polymerase, Bacillus stearothermophilus (Bst) DNA polymerase, Thermoanaerobacter thermohydrosulfuricus (Tts) DNA polymerase (U.S. Pat. No. 5,744,312), and the DNA polymerases of Thermus aquaticus, Thermus flavus or Thermus thermophilus. Equally preferred are the .φ0.29-type DNA polymerases, which are chosen from the DNA polymerases of phages: .φ0.29, Cp-1, PRD1,. φ0.15, .φ0.21, PZE, PZA, Nf, M2Y, B103, SF5, GA-1, Cp-5, Cp-7, PR4, PR5, PR722, and L17. In a specific embodiment, the DNA polymerase is bacteriophage .φ0.29 DNA polymerase wherein the multiple primers are resistant to exonuclease activity and the target DNA is linear DNA, especially high molecular weight and/or complex linear DNA, genomic DNA, cDNA.

Strand displacement during amplification, especially where duplex target templates are utilized as templates, can be facilitated through the use of a strand displacement factor, such as a helicase. In general, any DNA polymerase that can perform amplification in the presence of a strand displacement factor is suitable for use in the processes of the present invention, even if the DNA polymerase does not perform amplification in the absence of such a factor. Strand displacement factors useful in amplification include BMRFI polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehman, J. Virology 67(2):711-715 (1993); Skaliter and Lehman, Proc. Natl, Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out amplificaiton can be determined by testing the polymerase in a rolling circle replication assay such as those described in Fire and Xu, Proc. Natl. Acad. Sci. USA 92:4641-4645 (1995) and in Lizardi (U.S. Pat. No. 5,854,033, e.g., Example 1 therein).

In separate and specific embodiments, the target DNA may be, for example, a single stranded bacteriophage DNA or double stranded DNA plasmid or other vector, which is amplified for the purpose of DNA sequencing, cloning or mapping, and/or detection. The examples below provide specific protocols but conditions can vary depending on the identity of the DNA to be amplified and analyzed or sequenced.

The present invention relates to the ability to change the physical properties, particularly the Tm or melting temperature of the product DNA by changing the nucleotides used during amplification. In fact, amplification of the target template is not strictly required, merely replicating it with changed physical properties would be sufficient for some applications, but for most practical applications where amplification is desirable anyway, we refer to this step as “amplification”.

In U.S. Pat. No. 6,323,009 (see also U.S. patent application Ser. No. 09/920,571), a means of amplifying target DNA molecules is described. Some embodiments of this method feature the use of random-sequence hexamer primers added in great excess to target DNA, Φ29 DNA polymerase and the four normal dNTPs (dATP, dCTP, dGTP and dTTP) to produce multiple copies of all the sequences present in the original target sample. One way of checking that the product is similar to the starting target is to measure the Tm of both the product and the starting target template. Another is to use restriction endonucleases to digest the product DNA and the original target DNA and compare the sizes of the digestion products by gel electrophoresis. Similarly, sequence analysis can be performed on both the target and product DNAs.

In cases where such comparisons have been made, the Tm, restriction digest and sequence information clearly indicated that the product DNA is the same as the starting target DNA in the parameters that can usually be measured by these methods. Thus, while the overall molecular size of the product DNA may be much larger than the starting target DNA, its restriction digestion pattern, melting temperature and sequence are the same.

We found, however, that despite the consistent high quality and purity of the DNA produced by this amplification method, there remained some products that resisted sequence analysis, producing characteristic sequence patterns that stopped at repeat regions. These sequences similarly failed when the DNA was amplified by alternative means such as by growing larger quantities of culture and directly purifying the DNA from the host bacteria without amplification.

We also found that using modified reaction temperatures and times, amplification of template DNA could be carried out using analogs of the normal nucleotides, even when the normal nucleotide such as dGTP was completely replaced by an analog such as dITP. This results in remarkable amplification products that have Tm values that can be up to 26° C. lower than DNA made with the normal nucleotides. This is equivalent to the change in melting temperature expected by the addition of 40% formamide to the solvent,—that is a very strong denaturing condition.

While we have found that DNA sequencing of certain types of templates is improved by the methods of the present invention, this is just one example of an analysis method that relies on the hybridization of nucleic acid strands for its functionality. During the sequencing process, the primer must hybridize with its template, and the newly-synthesized strand must remain hybridized with its template strand in order to give a useful result. Many other methods of analysis rely on hybridization steps. These include hybridizations performed on solid surfaces such as Southern- and Northern-hybridizations, hybridizations on arrays and micro-arrays. They also include amplification by polymerase chain reaction (PCR) which itself can be used for genotyping and other analyses. Hybridization can also include self-hybridization to form intramolecular secondary structures (e.g. “hairpin” structures) such as those sensed by the SSCP analysis method. Even some forms of nuclease digestion such as digestion with restriction enzymes or RNAse H rely on hybridization of nucleic acid strands as part of the overall analysis process. Thus while some embodiments of this invention feature sequence analysis, the application of this invention is more broadly described as any process that comprises the use of modified MPA combined with an analysis method that relies on hybridization of nucleic acid strands generally.

In carrying out the procedures of the present invention it is to be understood that reference to particular buffers, media, reagents, cells, culture conditions, pH and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein. The invention is further described by reference to the examples below.

### EXAMPLES

The following examples present certain preferred embodiments of the instant invention but are not intended to be illustrative of all embodiments. These examples should not be construed as limiting the appended claims and/or the scope of this invention.

### Example 1

Sequencing Template DNA Made by Standard or Modified Multiply-Primed Amplification

a) Standard Multiply-Primed Amplification (MPA)

Amplification was carried out starting with 2 ng of double-stranded plasmid DNA (for example pNASS β DNA from Clonetech; Genbank XXU02433) in a 20 μl reaction volume containing 50 mM Tris-HCl, pH 8.25, 10 mM MgCl₂., 0.01% Tween-20, 75 mM KCl, 0.2 mM dATP, 0.2 mM dTTP, 0.2 mM dCTP and 0.2 mM dGTP, 100 pmoles (200 ng) of random hexamer and 100 ng φ 29 DNA polymerase. The reaction mixture was incubated at 30° C. for 16 hours to allow amplification of the DNA, and then incubated at 65° C. for 10 minutes to inactivate the polymerase. Typical yield is 2-4 μg of DNA product as measured by fluorescence assay using Picogreen dye (Molecular Probes).

b) Modified Multiply-Primed Amplification (mMPA)

The above standard amplification reaction was modified by omitting 0.2 mM dGTP and substituting 0.4 mM dITP alone or a mixture of 0.8 mM dITP and 0.05 mM dGTP. Plasmid DNA (2 ng pNASSβ) was amplified in a 20 μl reaction containing 50 mM Tris-HCl, pH 8.25, 10 mM MgCl₂., 0.01% Tween-20, 75 mM KCl, 0.2 mM dATP, 0.2 mM dTTP, 0.2 mM dCTP and 0.4 mM dITP or 0.8 mM dITP and 0.05 mM dGTP, 100 pmoles of random hexamer and 100 ng φ 29 DNA polymerase. The reaction was incubated at 30° C. for 16 hours to allow amplification of the pNASSβ DNA, and then incubated at 65° C. for 10 minutes to inactivate the polymerase. Typical yield with dITP alone is 0.1-0.3 μg of DNA product as measured by fluorescence assay using Picogreen dye (Molecular Probes). Typical yield with a mixture of dITP and dGTP is 1-2 μg of DNA. Yields are not corrected for possible differences in dye binding, but OD₂₆₀ readings in separate experiments suggest yields are fairly accurate. In all cases, the amount of DNA produced was more than required for multiple sequence analyses.

c) DNA Sequencing

The sequence of MHXP primer (specific for pNASSβ DNA) is 5′ ATTTCAGGTCCCGGATCCGGTG 3′ (SEQ ID NO: 1). 5 μl of each amplification reaction was transferred to a sequencing reaction mixture containing 5 pmoles of MHXP primer, and 8 μl of DYEnamic ET terminator premix (Amersham Biosciences) and water to a total volume of 20 μl. Reaction mixtures were cycled through 95° C., 20 seconds; 50° C., 30 seconds; and 60° C., 60 seconds, repeated 30 times. Reactions were then held at 4° C. until purification and analysis which was performed according to the manufacturer's instructions.

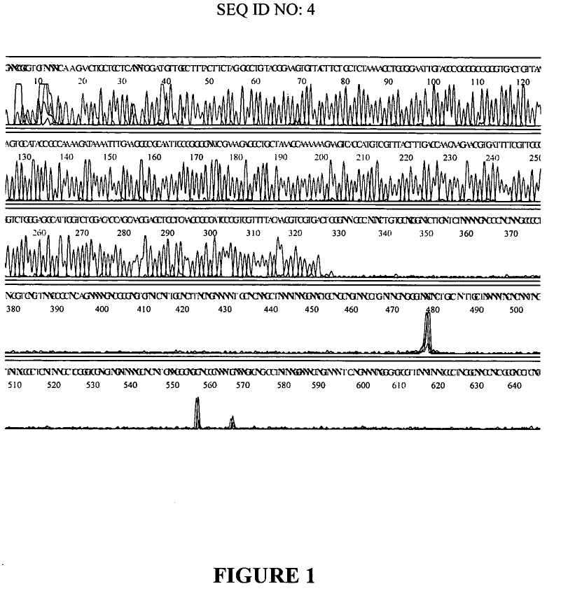

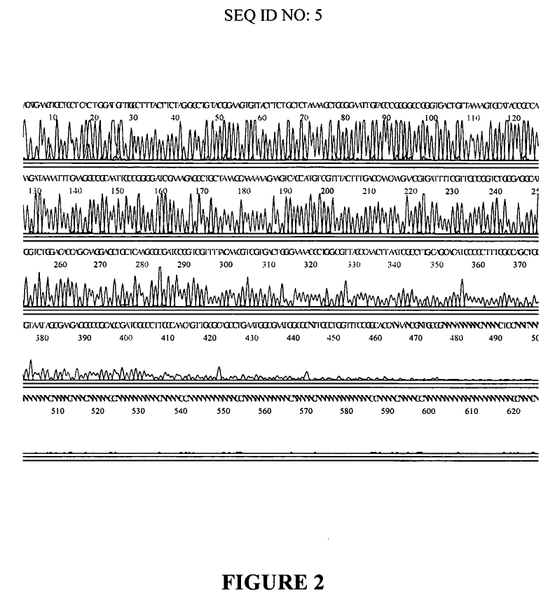

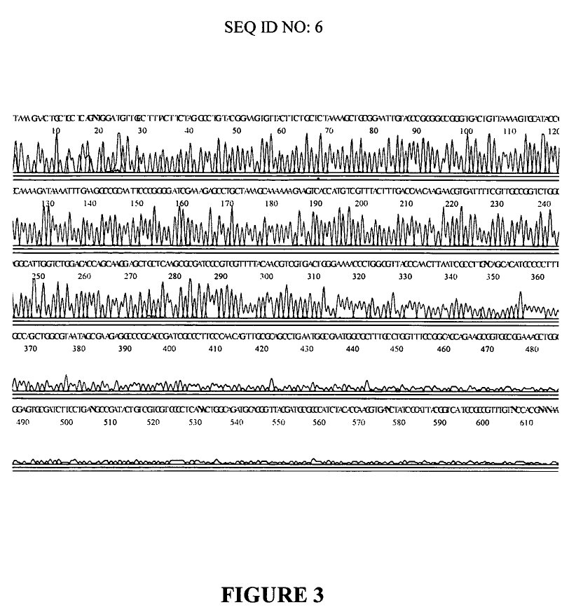

The samples were run on an ABI 3100 capillary sequencing instrument. The resulting electropherogram using Standard Multiply-Primed Rolling Circle Amplification on pNASSβ is shown in FIG. 1. The sequence obtained was accurate to about 400 nucleotides with a large reduction in signal intensity occurring between bases 310 and 320 (a “stop”). The DNA sequencing electropherogram using Modified Multiply-Primed Rolling Circle Amplification (with dITP alone) is shown in FIG. 2. The sequence obtained was accurate to about 450 nucleotides and had relatively even intensity throughout (no “stop”). Similar results are obtained using a mixture of 0.8 mM dITP and 0.05 mM dGTP during amplification (FIG. 3). In this case, the sequence obtained was accurate to at least about 600 nucleotides and had relatively even intensity throughout (no “stop”).

As can be seen, Modified Multiply Primed Amplification significantly improves the DNA sequencing result for this template.

### Example 2

The Melting Temperature (Tₘ) of DNA Amplified by Standard or Modified Multiply-Primed Amplification

DNA (plasmid pNASSβ) was amplified by Multiply-Primed Amplification with 0.2 mM dGTP (Standard) or 0.4 mM dITP or a mixture of 0.8 mM dITP and 0.05 mM dGTP as described in detail in Example 1. 20 reaction mixtures of 20 μl each were incubated at 30° C. for 16 hours, and then incubated at 65° C. for 10 minutes.

Each batch of 20 reactions was pooled together, precipitated by ethanol and resuspended in 400 μl of 1×SSC buffer (150 mM NaCl, 15 mM Na₃ Citrate). The OD at 260 nm was adjusted to be in the range of 0.2 to 0.5 using 1×SSC buffer in order to perform Tₘ measurements. The OD at 260 nm was measured as temperature changed from 30° C. to 98° C. using a Lambda 25 UVN is Spectrophotometer (Perkin Elmer Inc.). The Tₘ of the DNA was determined as the peak in the first derivative of the OD₂₆₀ vs temperature curve which is also approximately the temperature at which 50% of the total increase in OD₂₆₀ is observed. The Tₘ of pNASSβ DNA amplified with dGTP is 95° C. whereas the Tₘ of DNA amplified with dITP is 69° C. and that amplified with the mixture of dITP and dGTP is 75° C.

### Example 3

Reaction Products of Modified Multiply-Primed Amplification (mMPA) can be Cycle Sequenced at Lower Temperatures than Products of Standard Multiply-Primed Amplification (MPA).

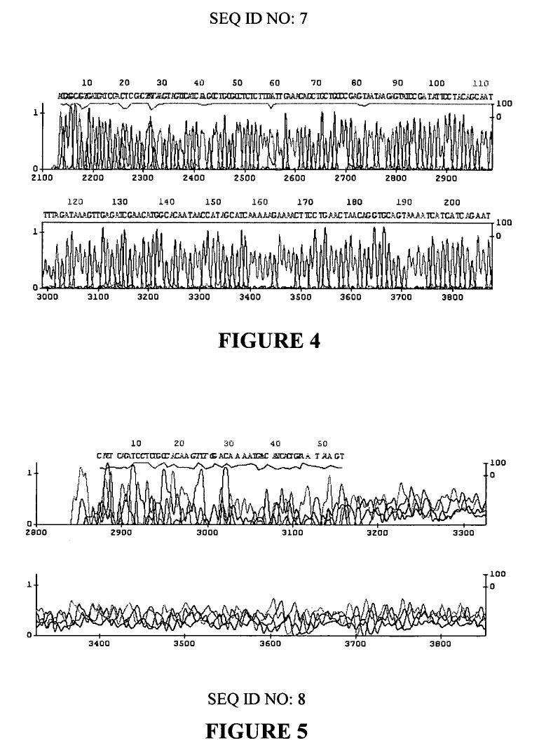

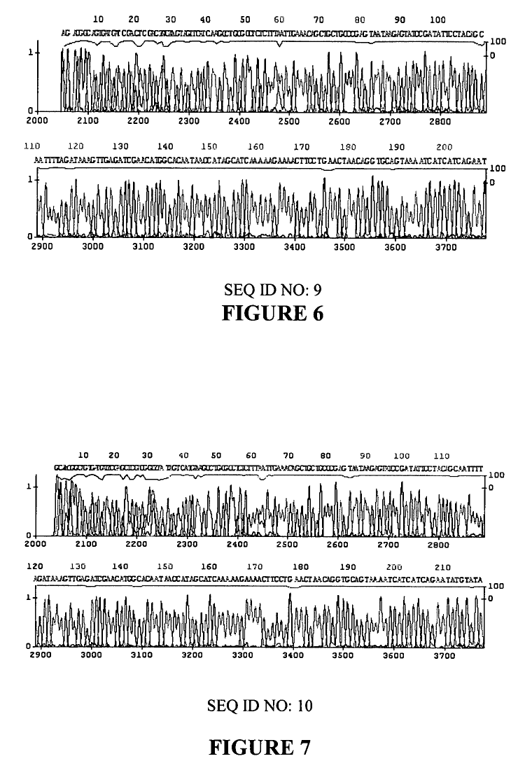

A randomly selected clone from a library of T. Volcanium DNA in pUC 18 was amplified by Standard (dGTP) Multiply-Primed Amplification or modified (a mixure of dITP and dGTP) Multiply-Primed Amplification as described in detail in Example 1. Then sequencing reactions were carried out using 5 pmoles of −40 Universal M13 primer and 8 μl of DYEnamic ET terminator premix and 5 μl of the amplified DNA. Reactions were cycled at normal temperatures (30 times at 95° C., 20 seconds, 50° C., 30 seconds and 60° C., 60 seconds) or at low temperatures (30 times at 82° C., 20 seconds, 40° C., 30 seconds and 50° C., 60 seconds). Samples were precipitated by ethanol, dissolved in 20 μl of 95% formamide and run on a MegaBACE 1000 capillary sequencing instrument (Amersham Biosciences). The electropherogram obtained with the dGTP-amplified clone is shown in FIGS. 4 (high temperature cycles) and 5 (low temperature cycles). Results from the dITP and dGTP-amplified clone are shown in FIGS. 6 (high temperature cycles) and 7 (low temperature cycles). The sequence obtained using standard amplification and low temperature cycling has very weak signal that is impossible for the instrument software to interpret.

As shown, only the products of the modified amplification reaction can be sequenced using the low temperature thermal cycles.

### Example 4

DNA Amplified by Modified Multiply-Primed Amplification has Altered Activity with Restriction Enzymes

Double-stranded pUC 19 DNA (2 ng, Amersham Biosciences) was amplified by Multiply-Primed Rolling Circle Amplification with dGTP (0.2 mM) or dITP (0.4 mM) as described in detail in Example 1. After incubation overnight at 30° C., 10 μl of each reaction mixture was digested with 5 units of HindIII for 2 hours at 37° C. in a 20 μl reaction volume containing 10 mM Tris-HCl (pH 8.0), 7 mM MgCl₂, 60 mM NaCl and 2 μg bovine serum albumin. An additional 10 μl of the each reaction product was also digested with 5 units of BamHIII for 2 hours at 37° C. in a 20 μl volume containing 10 mM Tris-HCl (pH 7.5), 7 mM MgCl₂, 150 mM KCl and 2 μg bovine serum albumin. The products of modified and standard amplified pUC19 DNA along with the digestions were electrophoretically separated on a 1% agarose gel in 1×TBE buffer (89 mM Tris base, 89 mM boric acid, 2 mM EDTA, pH 8.3). Both the starting pUC19 and pUC19 amplified under standard conditions can be cut by either BamHI or HindIII. DNA prepared by modified (dITP) amplification is cut by HindIII (AAGCTT) (SEQ ID NO: 2) but not by BamHI (GGATCC) (SEQ ID NO: 3). Knowing that some restriction endonucleases tolerate substitution of dI for dG (Modrich P, Rubin R A. J Biol. Chem. 1977 252 7273-8) but BamHI, in particular, does not (Kang Y K et. al Biochem Biophys Res. Comm. 1995 206:997-1002), this suggests that dG is indeed replaced by dI in modified amplification products.

### Example 5

Use of DNA Polymerase variants for Modified Multiply-Primed Amplification (mMPA)

2 ng pUC19 DNA was amplified by Multiply-Primed Amplification with dGTP (0.2 mM) or dITP (0.4 mM) or mixture of dITP (0.8 mM) and dGTP (0.5 mM) as described in detail in Example 1 using 100 ng of the wild type Phi 29 DNA polymerase and each of the following variants with single amino acid substitutions: N62E, N62D, DI 2A, E14A, D66A and D169A (Bernad A, Blanco L, Lazaro J M, Martin G, Salas M., Cell 1989 59:219-28 and Esteban J A, Soengas M S, Salas M, Blanco L., J Biol Chem 1994 269:31946-54). The reactions were incubated at 30° C. for 16 hours, and then incubated at 65° C. for 10 minutes. The Picogreen dsDNA quantitation Kit (Molecular Probes Inc) was used to quantify the product DNA using bacteriophage lambda DNA as standard. The resulting DNA yields are shown in Table 1.

TABLE 1

| DNA | DG amplified | dI amplified | DI + dG amplified |
|---|---|---|---|
| Polymerase | DNA (μg) | DNA (μg) | DNA (μg) |
| Wild Type φ 29 | 16 | 0.15 | 0.74 |
| N62E φ 29 | 30 | 1.85 | 2.95 |
| N62D φ 29 | 31 | 2.13 | 3.71 |
| D12A φ 29 | 43 | 0.17 | 0.18 |
| E14A φ 29 | 44 | 0.14 | 0.17 |
| D66A φ 29 | 68 | 0.07 | 0.10 |
| D169A φ 29 | 73 | 0.15 | 0.16 |
| φ DNA polymerase variants N62E and N62D give about ten-fold higher yield of amplified DNA using dITP only modified amplification than wild-type φ 29. With the mixture of dITP and dGTP, these variants yield about 4-5 fold more product than wild-type φ 29. DNA amplified using these polymerase variants appeared to have | | | |
| # similar size distribution as DNA amplified using wild-type polymerase and gave similar results when used as template for DNA sequencing experiments. | | | |

Those skilled in the art having the benefit of the teachings of the present invention as set forth above, can effect numerous modifications thereto. These modifications are to be construed as being encompassed within the scope of the present invention as set forth in the appended claims.

1. A method for amplifying nucleic acid sequences, comprising:
a) forming a mixture containing multiple single stranded oligonucleotide primers, one or more amplification targets, a DNA polymerase and multiple deoxynucleoside triphosphates
wherein one or more of the deoxynucleoside triphosphates is a modified deoxynucleoside triphosphate that upon incorporation changes the melting temperature (Tm) of amplified DNA products by at least 1° C. from the melting temperature (Tm) of said one or more amplification targets; and
b) incubating said mixture under conditions wherein said one or more amplification targets bind to more than one of said primers to promote replication of said one or more amplification targets by extension of primers to form multiple amplified DNA products.

2. The method of claim 1 wherein said one or more modified deoxynucleoside triphosphates upon incorporation, change the melting temperature (Tm) of the amplified DNA products by at least 3° C.

3. The method of claim 1 wherein the one or more modified deoxynucleoside triphosphates upon incorporation, change the melting temperature (Tm) of the amplified DNA products by at least 5° C.

4. The method of any one of claims 1-3 further comprising:
hybridizing the amplified DNA products containing one or more modified nucleotides with one or more oligonucleotides or hybridization probes for sequence-based analysis, indicating either the presence or extent of hybridization.

5. The process of any one of claims 1-3 further comprising:
hybridizing the amplified DNA products with a sequencing primer and sequencing, or cycle sequencing, the amplified DNA products by the dideoxy chain-termination method.

6. A method of changing the susceptibility of DNA to cleavage by restriction enzymes comprising modifying the one or more restriction sites present in said one or more amplification targets by amplifying said one or more amplification targets in the presence of one or more modified deoxynucleoside triphosphates according to the method of any one of claims 1-3.

7. A method of modifying the single-strand conformational properties of one or more amplification targets by amplifying said targets using one or more modified deoxynucleoside triphosphates according to the method of any one of claims 1-3.

8. A method of detecting a single base substitution comprising modifying the single-strand conformational properties of a one or more amplification targets according to the method of claim 7 and analyzing the amplified DNA products by electrophoresis under conditions suitable for single-strand conformational polymorphism analysis.

9. The method of any one of claims 1-3, wherein the said modified deoxynucleoside triphosphate is selected from the group consisting of dITP, 7-deaza-dGTP, 7-deaza-dITP, 7-substituted-7-deaza-dITP, 7-substituted-7-deaza-dGTP, 7-deaza-dATP or related analogs, N4-alkyl-dCTP, 5-alkyl-dCTP or related analogs, and 5-substituted dTTP.

10. The method of claim 9, wherein the said modified deoxynucleoside triphosphate is dITP.

11. The method of any one of claims 1-3, wherein the polymerase is a Φ29 type DNA polymerase.

12. The method of claim 11, wherein the polymerase is selected from wild-type, N62E or N62D variants of Φ 29 DNA polymerase.

13. The method of any one of claims 1-3, wherein the resultant amplified DNA products are susceptible to cleavage by hydrolytic enzymes that do not cleave the original said one or more amplification targets.

14. The method of claim 13, wherein the hydrolytic enzyme is a nicking enzyme.

15. The method of claim 13 wherein the hydrolytic enzyme is a glycosidase.

16. A kit for amplyfing nucleic acid sequences comprising multiple single stranded oligonucleotide primers, a DNA polymerase and one or more modified nucleoside triphosphates that upon incorporation changes the melting temperature (Tm) of amplified DNA products from the melting temperature (Tm) of said one or more amplification targets.

17. A kit for sequencing a nucleic acid comprising multiple single stranded oligonucleotide primers, a DNA polymerase and one or more modified nucleoside triphosphates that upon incorporation changes the melting temperature (Tm) of amplified DNA products from the melting temperature (Tm) of said one or more amplification targets, a DNA polymerase suitable for dideoxy chain-termination DNA sequencing, deoxynucleoside triphosphates, and at least one chain-terminating dideoxynucleoside triphosphate.
